(19) United States
(12) Patent Application Publication
(10) Pub. No.: US 2003/0191301 A1
(43) Pub. Date: Oct. 9, 2003

(54) CLONING OF A HIGH GROWTH GENE

(76) Inventors: JUAN F. MEDRANO, DAVIS, CA (US); SIMON HORVAT, EDINBURG (GB); ERIC BRADFORD, DAVIS, CA (US)

Correspondence Address: QUINE INTELLECTUAL PROPERTY LAW GROUP, P.C., P O BOX 458, ALAMEDA, CA, 94501, US

(21) Appl. No.: 08/999,477

(22) Filed: Dec. 29, 1997

(51) Int. Cl. C07H 21/02; C07H 21/04; C12P 21/06; C12N 15/09; C12N 1/00; C12N 15/00; C12N 15/63; C12N 15/70; C12N 15/74; C12N 5/00

(52) U.S. Cl. 536/23.51; 435/69.1; 435/320.1; 435/243; 435/325; 536/23.1

(57) ABSTRACT

A mouse cDNA is illustrated by FIG. 3 (SEQ ID NO:1) which, when knocked out or prevented from expression, results in high growth. This mouse cDNA is highly homologous to genes of other species, such as human and cattle.

This invention was made with Government support under Grant Nos. HD 00394 and HD 07205, awarded by the National Institutes of Health and Grant No. 92-37205-7840 awarded by the United States Department of Agriculture. The Government has certain rights in this invention.

### FIELD OF THE INVENTION

The present invention generally relates to modifying growth in domestic animals, and more particularly to oligonucleotide probes useful to isolate genomic clones so as to improve growth performance and efficiency in domestic animals, such as by knocking out loci related to the control of growth or utilizing identified growth quantitative trait loci in marker-assisted selection programs with domestic animals.

### BACKGROUND OF THE INVENTION

In domestic animals, many different hormones (e.g. growth hormone, sex steroids) are known to have an important function in maintaining normal animal growth and to be effective growth promoters when administered exogenously (e.g. Kopchick, Livestock Prod. Sci., 27:66-75, 1991). Some transgenic animals have been caused to express a growth hormone, and increased growth of such transgenic animals has been reported. Palmiter et al. (Nature, 300:611, 1982) microinjected the male pronucleus of fertilized mouse eggs with a DNA fragment containing the promoter of the mouse metallothionein-I gene fused to the structural gene of rat growth hormone. Several of the transgenic mice developed from the genetically modified zygote exhibited a growth rate substantially higher than that of control mice. Palmiter et al. (Science, 222:809, 1983) demonstrated that a similar enhancement of growth could be obtained in transgenic mice bearing an expressible human growth hormone gene. A like effect is observed when human growth hormone releasing factor is expressed in transgenic mice. (Hammer et al., Nature, 315:413, 1985). Bovine growth hormone has also been expressed in transgenic animals (McGrane et al., J. Biol. Chem., 263:1144351, 1988; Kopchick et al., Brazil. J. Genetics, 12:37-54, 1989).

U.S. Pat. No. 5,350,836, inventors Kopchick et al., issued Sep. 27, 1994, entitled “Growth Hormone Antagonists,” describes administration of a mutein that has growth-inhibitory activity in vertebrates and may be administered to mammals, such as bovines, when growth inhibition is desirable. Alternatively, a gene coding for the hormone is suggested for introduction into a prenatal form of a mammal to produce growth-inhibited animals.

A recent article discusses the biological function of a transforming growth factor β superfamily member and suggests it as a potentially useful target for genetic manipulation in cattle and other farm animals. This new member is called myostatin (“GDF-8”) and functions as a negative regulator of skeletal muscle growth. It was initially studied in gene knockout experiments in mice, followed by a report of the myostatin sequences of nine other vertebrate species and the identification of mutation in double-muscled cattle. McPherron et al., Nature, 387:83-90 (1997); McPherron and Lee, PNAS, 94:12457-12461 (1997). In mice, myostatin knockouts were significantly larger than normal mice and showed a large increase in muscle mass. In Belgian Blue cattle a small deletion of eleven nucleotides and in Piedmontese cattle a single base pair mutation in the myostatin gene produced myostatin null animals having a characteristic increase in muscle mass known as “double muscling.”

A high growth, mutant mouse with unusually rapid weight gain is also known (Bradford and Famula, “Evidence for a major gene for rapid postweaning growth in mice,” Genet. Res., 44:293-308, 1984). The mutation was reported to be a segment of DNA located in mouse chromosome 10 that was deleted. (Medrano et al., “Growth hormone and insulin-like growth factor-I measurements in high growth (hg) mice,” Genet. Res., 58:67-74, 1991; Medrano et al., “The high growth gene (hg) in mice is located on chromosome 10 linked to Igf1. Advances in gene technology: Feeding the world in the 21st century,” edited by W. J. Whelan et al., The 1992 Miami Bio/Technology Winter Symposium, 1:12, 1992; Horvat and Medrano, “Interval mapping of high growth (hg), a major locus that increases weight gain in mice,” Genetics, 139:1737-1748, 1995; Horvat and Medrano, “The high growth (hg) locus maps to a deletion in mouse chromosome 10,” Genomics, 36:546-549, 1996.) The region of mouse chromosome 10 where the high growth gene was localized is homologous to a region in human chromosome 12, cattle and pig chromosome 5, and sheep chromosome 3. The high growth mouse phenotype features of interest are: a 30-50% increase in growth of tissues and organs, but where growth does not result in obesity; an increase in the efficiency of conversion of feed to muscle mass; decreased growth hormone levels in pituitary and plasma; an elevated plasma level of insulin growth factor-1; and, an increased muscle mass due primarily to an increase in muscle fiber number (i.e., hyperplasia) and a moderate fiber hypertrophy.

Control of growth for higher organisms has a number of applications. For example, with domestic species the characterization of the gene or genes causing high growth phenotype should offer new ways to improve growth performance and efficiency. In some human growth disorders, it has been suggested that as yet unknown genetic factor(s) may be at work (Jones, K. L., “The etiology and diagnosis of overgrowth syndromes,” Growth Genetics and Hormones, 10:6-10, 1994). A marker closely linked to a growth disorder would be useful in diagnosis and genetic counseling, and the development of a treatment to suppress or enhance genetic growth action would be useful.

### SUMMARY OF THE INVENTION

In one aspect of the present invention, an isolated nucleic acid molecule is provided that encodes a gene product which, when knocked-out, results in a high growth phenotype. For example, a mouse cDNA is provided which is highly homologous to genes of various species, such as mouse, bovine, and human, which are related to a high growth phenotype. The mouse cDNA is shown as SEQ ID NO:1. The present invention provides for cloning of this gene and biologically active fragments thereof, as well as preparation of oligonucleotide probes, or primers. These are useful in identifying molecules and pathways of growth regulation so as to improve animal growth and to design diagnostic and treatment strategies for growth disorders, and to develop genetic markers. Monoclonal or polyclonal antibodies are further provided to protein products of the high growth locus, which are useful for affinity purifications and diagnostic assays for high growth family members.

### BRIEF DESCRIPTION OF THE DRAWINGS

FIG. 1 illustrates a physical map of the high growth (“hg”) region where: (A) are polymerase chain reaction (PCR)-based markers (sequence tagged sites, STSs) from ends of clones shown non-italicized, the genetic (microsatellite) marker “D10Mit69” is italicized, and a PCR-based marker derived from an exon trapping product we hereinafter call “B308A” is typed in bold; (B) are genomic DNA of control mice tested as progenitors of high growth mice, AKR/J, C3H/HeJ, C57BL/6J, and DBA/2J, with the high growth mouse line being tested being C57BL/6J-hghg; (C) are Yeast Artificial Chromosome (YAC) clones, and (D) are Bacterial Artificial Chromosome (BAC) clones;

FIG. 2 illustrates Northern blots with hybridization to mouse embryonic stages and adult mouse tissues using the candidate exon “B308A” as a probe (two upper panels), and as a control the bottom two panels are blots stripped off the “B308A” probe and reprobed with cDNA for human B-actin gene;

FIG. 3A is the nucleotide sequence of the cDNA in the inventive mouse clone called “B308A-6-1” (SEQ ID NO:1);

FIG. 3B is the protein translation of the B308A-6-1 coding sequence (SEQ ID NO:4);

FIG. 4 is the nucleotide sequence of the original consensus B308 exon that was isolated, where polymorphism (A or T) found in one clone is indicated by a bold underlined T in position 286, and where primers used with this sequence are indicated with arrow lines (SEQ ID NO:2);

FIG. 5 is a bovine fragment of the hg gene (SEQ ID NO:3) obtained with PCR primers of the inventive cDNA mouse clone;

FIG. 6 is a diagram of gene knock-out experiment for identification of the high growth gene, or locus; and

FIG. 7 is the identification of high growth by gene addition.

### DETAILED DESCRIPTION OF THE PREFERRED EMBODIMENT

SEQ ID NO:1 illustrates a mouse cDNA, which is the first and only candidate gene in the high growth (“hg”) region believed to be discovered to date. The hg region appears to be highly conserved, as will be more fully described herein.

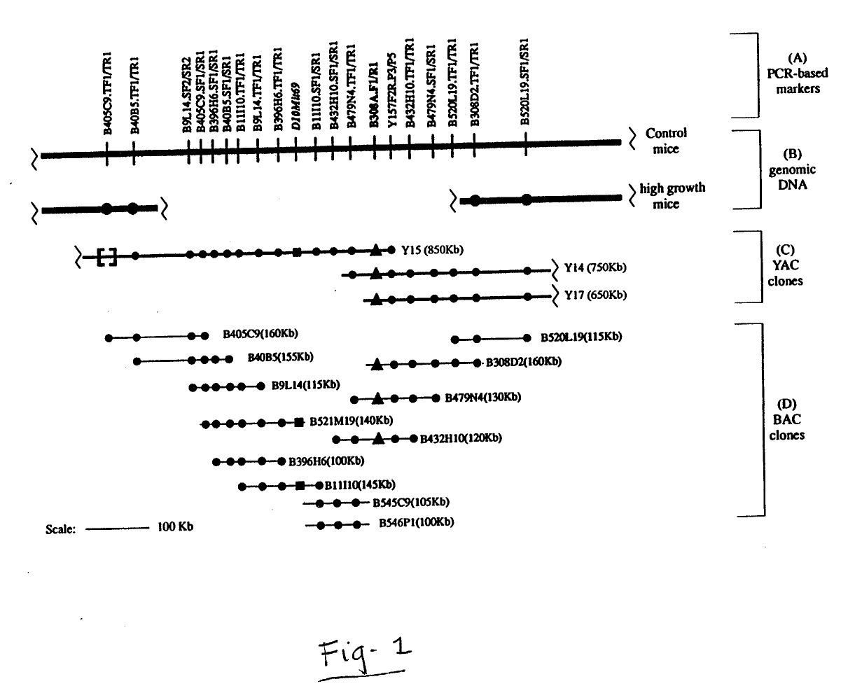

Turning to FIG. 1, the deleted microsatellite marker, D10Mit69, was utilized as an entry point to physical cloning of the hg-containing segment using Yeast Artificial Chromosome (YAC) and Bacterial Artificial Chromosome (BAC) cloning systems. The size of the deletion in high growth mice, estimated from the clone lengths, is on the order of a half million base pairs.

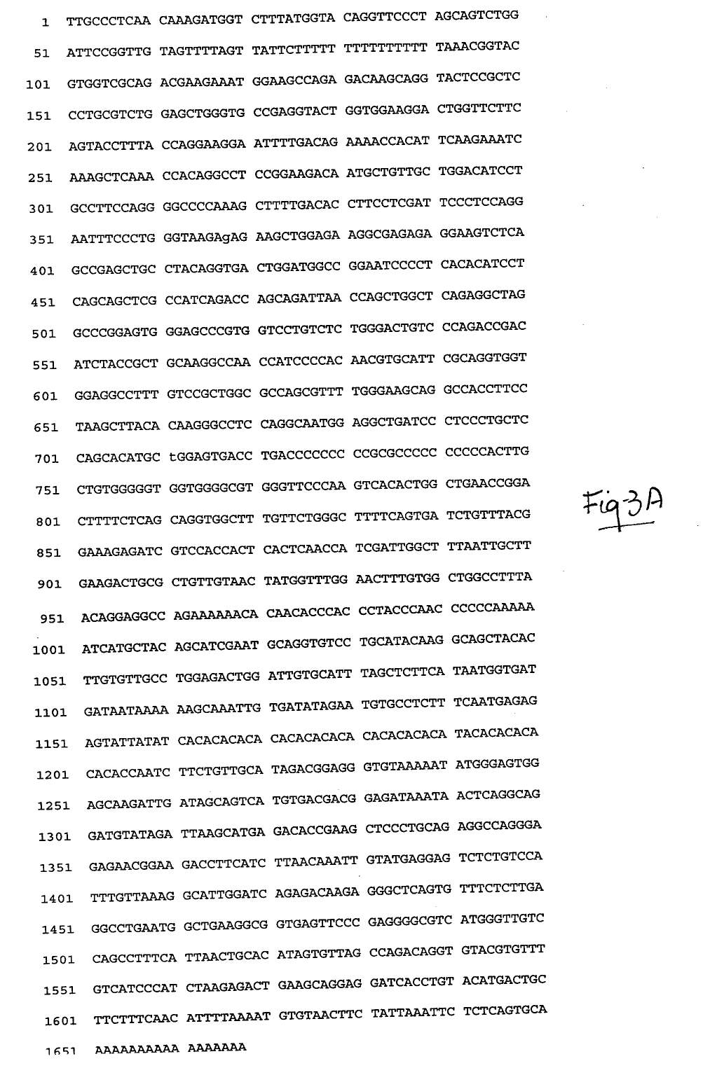

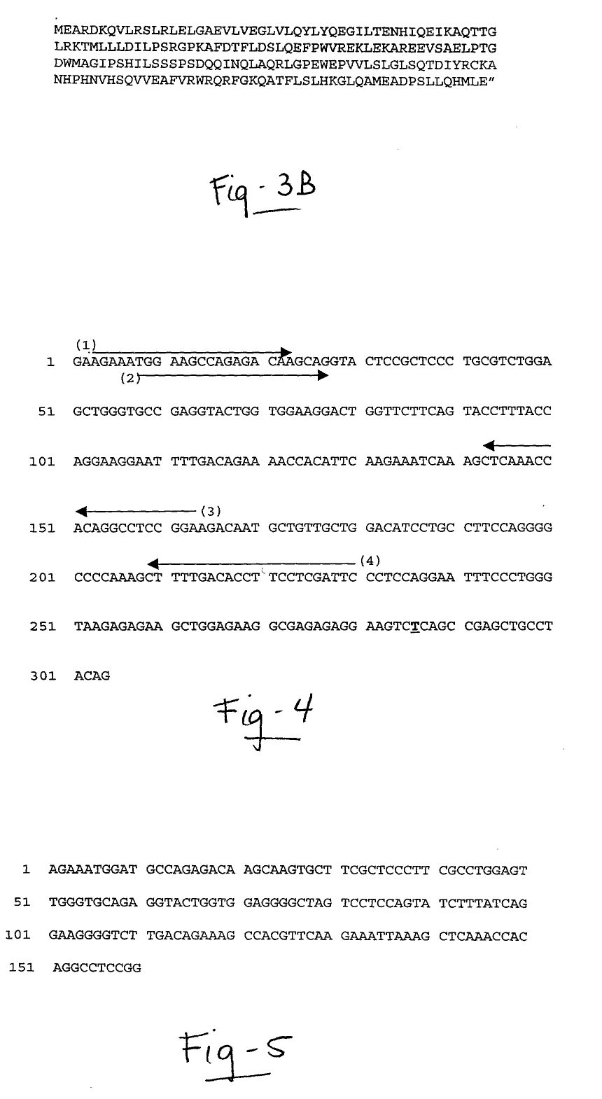

The open reading frame of the mouse B308A-6-1 (FIG. 3A) is predicted to encode 199 amino acids (FIG. 3B) which share very high homology (178/199 identical amino acids) with the human protein. The nucleotide sequence of the original exon-trap clone, with the position of primers 1, 2, 3, and 4 is indicated by FIG. 4 (this partial sequence is SEQ ID NO:2). The bovine sequence, which is yet a partial coding sequence, is SEQ ID NO:3 and is shown by FIG. 5. This PCR amplified sequence was from reverse transcribed lactating mammary gland mRNA using the mouse primers 2 and 3 (the primers indicated by FIG. 4).

There is high homology observed between the mouse and bovine sequences (49/52 identical amino acids). The homology between the mouse protein discussed here and the human RAIDD proteins reported by another group (Duan and Dixit, “RAIDD is a new ‘death’ adaptor molecular,” Nature, 385:86-89, 1997) is very conserved when compared in the conserved, NH₂ and C terminal domains. In the NH₂-terminal domain (amino acids 1-80), 79 out of 80 residues are identical, whereas in the C-terminal “death domain” (amino acids 123-199), 77 out of 86 amino acids are identical. Therefore PCR primers in these domains should be very useful to pick up the homologous DNA segments in other species. The bovine B308A segment, SEQ ID NO:3, was obtained using mouse primers on the NH₂-terminal domain.

Without being bound by theory, we suggest that high growth mice are bigger because they have more cells that are moderately larger. The control of cell number depends primarily on the balance between processes of proliferation and cell death (apoptosis) (Jacobson et al. “Programmed cell death in animal development,” Cell, 88:347-354, 1997; Raff, “Size Control—The Regulation of Cell Numbers in Animal Development,” Cell, 86:173-175, 1996). In mammals, apoptosis begins at blastula stage and continues throughout life and can be of equal importance in controlling cell numbers as cell proliferation (Raff, supra). There are nematode mutations that abolish apoptosis and result in a worm with a 15% increase in cell numbers, a normal lifespan, morphology and behavior (Ellis and Horvitz, “Genetic-control of Programmed Cell-death in the Nematode C-elegans,” Cell, 44:817-829, 1986). It might be possible therefore that the high growth phenotype as a result of an increase in cell numbers might be due to a perturbed apoptosis program caused by a lack of function of a apoptotic protein such as that corresponding to our clone B308A-6-1, which is homologous to human RAIDD (Duan and Dixit, supra).

Returning to FIG. 1, we have cloned a high growth (“hg”) region in bacterial artificial chromosomes (“BAC”) and yeast artificial chromosomes (“YAC”). Marker D10Mit69 was used to initiate the bi-directional chromosomal walk. A BAC library (Research Genetics, Huntsville, Ala., USA) was screened as follows: so-called higher-order pools containing DNAs from several 384-clone plates were screened by polymerase chain reaction (PCR) to identify a positive 384-well plate. Clones from this plate were then grown on membranes, colony-lysed (Nizetic et al., “Construction, arraying, and high density screening of large insert libraries of human chromosomes X and 21: their potential use as reference libraries,” Proc. Natl. Acad. Sci. USA, 88(8):3233-3237, 1991) and hybridized to a relevant probe. If a probe was a microsatellite marker (such as D10Mit69) or contained other types of repetitive DNA, an oligo probe was designed in the unique parts of the marker to prevent cross hybridization to clones containing these repeats. Identified single positive BAC clones were sized on a pulsed-field gel apparatus (CHEF-DRIII, Bio-Rad) and sequenced from the ends of the insert (Wang et al., “Construction and characterization of a human chromosome 2-specific BAC library,” Genomics, 24:527-534, 1994). These sequences were utilized to construct a PCR primer pair at each end (so-called sequence tagged sites, “STS,” which were in turn used to screen the BAC library again to isolate the next overlapping clone(s). Each end STS was examined for amplification in hg mouse and its parental strains to test whether a deletion breakpoint had been crossed. The cloning of the hg region in BAC clones was complete once clones that span the whole deletion and both deletion endpoints were obtained. A map of YAC and BAC clones in the hg region is illustrated by FIG. 1.

BAC clones B308D2 and B11I10 (FIG. 1) were subcloned in vector pSPL3 (GibcoBRL, New York, USA) which flanks an insert with splice donor and splice acceptor sites. These pSPL3 subclones of BACs were transfected into COS7 cells (African-green monkey cells obtained from American type culture Collection, Maryland, USA) using electroporation following manufacturer's protocols (BioPulser, Bio-Rad, California, USA). RNA was isolated from cell cultures 24 hours following the transfection using Trizol regent (Gibco-BRL). Reverse transcription and PCR amplification were as described in Church et al., “Isolation of genes from complex sources of mammalian genomic DNA using exon amplification,” Nat. Genet., 6:98-105, 1994. Each exon trapping product was then cloned (TA cloning kit, Invitrogen, USA) and used as a probe in hybridizations to blots of BAC digests to verify whether the exon trapping product was derived from a particular BAC(s). Candidate exon trapping products were then sequenced.

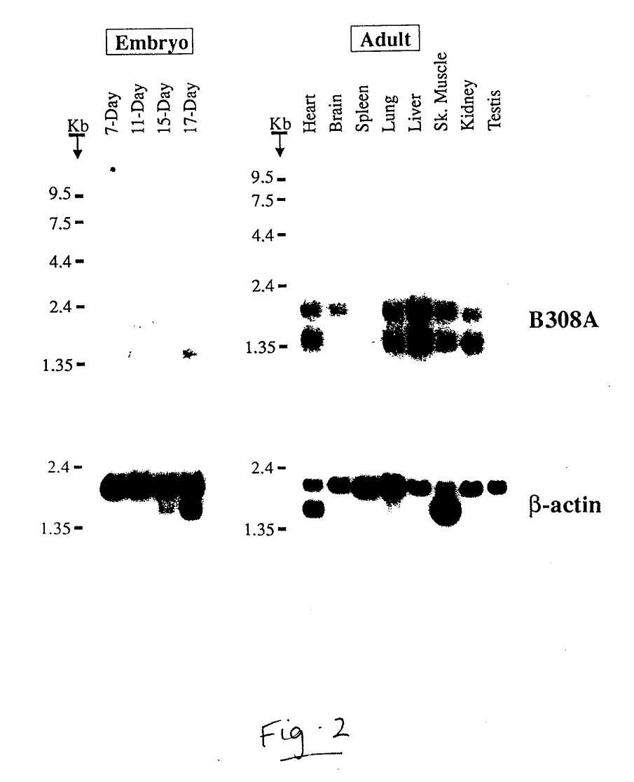

The sequence of end STSs and candidate exon trapping products were compared to all sequences in public sequence data banks with BLAST and FASTA computer programs to test for similarity to known genes or expressed sequence tags (ESTs). Candidate exons were then screened for the presence of corresponding RNA from a variety of tissues and developmental stages using Northern blots. The sequence of the candidate exon trapping product B308A was found to be highly homologous to an EST derived from the mouse embryo (93% identity) and several human ESTs (83-87% identity) derived from fetal liver/spleen and infant brain, and to the human death adaptor molecule (86% similarity), RAIDD (GenBank Accession No. U79115) (Duan and Dixit, supra) or CRADD (GenBank Accession No. U84388) (Ahmad et al., “CRADD, a novel human apoptotic adaptor molecule for caspase-2, and FasL/Tumor necrosis factor receptor-interacting protein RIP,” Cancer Research, 57:615-617, 1997). The Northern analysis showed that the RNA containing exon trapping B308A sequence is widely expressed in several tissues and developmental stages, most notably in liver (FIG. 2).

Using the candidate exon B308A as a probe, a mouse mammary (15-day gestation) cDNA library (C. Watson, Roslin Institute, personal communication) was screened using standard procedures for lambda phage cDNA library screens (Maniatis et al., “Molecular Cloning: A Laboratory Manual,” Cold Spring Harbor Lab., Cold Spring Harbor, N.Y., 1992). A total of 11 lambda phage cDNA clones were isolated—one clone of ˜1.6 Kb and 10 clones of ˜1 Kb. The ˜1.6 Kb clone (clone B308A-6-1, FIG. 3) was then thoroughly sequenced using primer walking method. B308A-6-1 was also mapped back to the hg deletion (FIG. 1). The cDNA containing B308A-6-1, SEQ ID NO:1, represents the first and only candidate gene in the hg region to date.

Using mouse PCR primers we have amplified and sequenced a fragment corresponding to the NH₂-terminal domain of B308A-6-1 in reverse transcribed RNA from cow lactating mammary gland (FIG. 5). A comparison between the predicted amino acid sequences of mouse, bovine, and human proteins shows that the sequence of B308A-6-1 is highly conserved between mouse, human, and cattle. This lends experimental support to the conservation of this region in other animals and suggests hg will most likely be found in the genome of other domestic animals, including poultry.

From a personal communication, a putative quantitative trait locus (qtl) for growth in a chicken cross has been mapped and the mapped region shown to have the hg gene. We propose to use the mouse cDNA, SEQ ID NO:1, to isolate a cosmid containing chicken hg in a collaborative effort. The hg homolog will be checked by DNA sequencing and the gene mapped by fluorescent in-situ hybridization (“FISH”) onto chicken metaphase spreads. Next, markers will be developed based on the chicken genomic sequence and mapped on to a reference genetic linkage map of chicken (the map being available at the Web site http://www.ri.bbsrc.ac.uk/). The hg marker will be used to genotype F2 progeny of the cross to confirm linkage to the growth QTL.

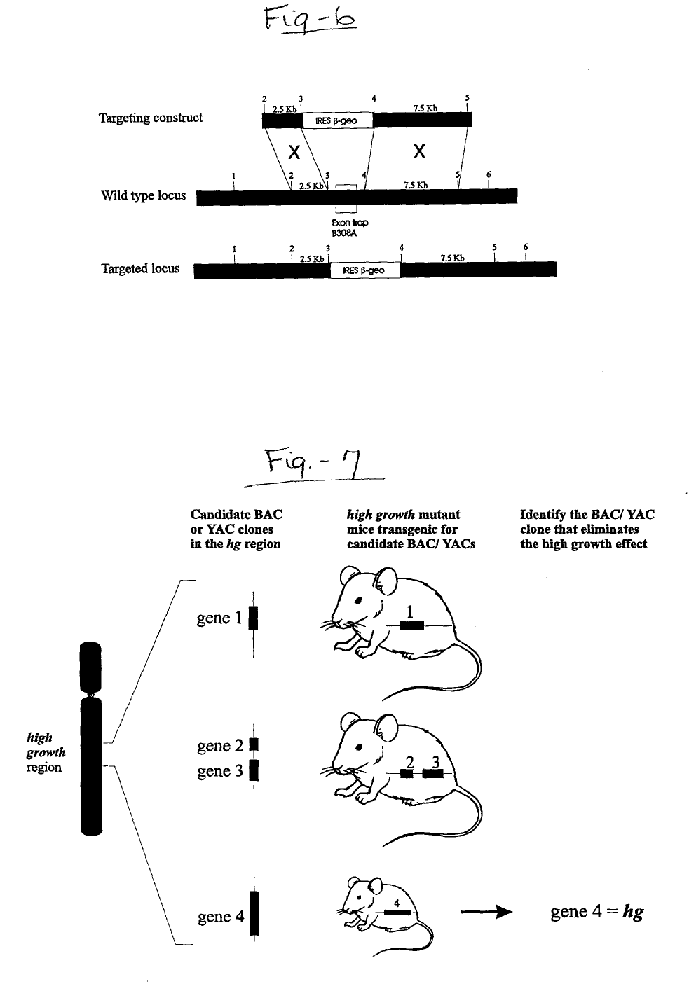

Since in mammals there is a gene about every 50 kb and a deletion in high growth mice is about 500 Kb, further transcript mapping may identify several additional genes in the hg region. The deletion in the hg region suggests that high growth effect is most likely due to a lack-of-function of hg. Therefore any gene that maps to a deletion is potentially a candidate for hg. We expect to conduct transgenic analysis for gene identification, as shown in FIGS. 6 and 7.

It should be feasible to identify cognate genes by in vivo complementation. The addition of wild type copies of the hg gene onto a high growth mutant background should eliminate the high growth effect. High growth mice transgenic for hg would be expected to grow more slowly than non-transgenic high growth mice. For this purpose, we plan to create transgenic mice containing candidate DNA constructs or candidate large insert clones such as YACs and BACs. Transgenic lines carrying these constructs will then be tested for their ability to complement the hg mutation in breeding studies.

Molecular cloning of hg will permit the functional characterization of hg and enable isolation and functional studies of the homologous gene in man and domestic animals. In humans, the hg gene can be tested for associations in various growth disorders, especially those whose etiology or map positions are unknown. If the association between hg and a growth disorder is established, it permits designing diagnostic and treatment strategies for such growth disorders. Manipulation of the hg gene may be applied in livestock, such as direct treatment with hg gene product (or against hg product) or using the gene transfer approach.

Methods enabling genetic manipulation of genes in livestock species are being developed, such as described by Wilmut et al., “Viable Offspring Derived from Fetal and Adult Mammalian Cells,” Nature, 385:810-813 (1997), so that application by hg knockout should prove effective in domestic animals to enhance growth. We also envision designing a ribozyme/anti-sense delivery system for transgenic animals to eliminate or reduce the effect (knockdown) of hg, by analogy to the work of L'Huillier et al., “Efficient and Specific Ribozyme-Mediated Reduction of Bovine α-Lactalbumin Expression in Double Transgenic Mice,” Proc. Natl. Acad. Sci. USA, 93:6698-6703 (1996). Also, Xie et al. have quite recently described a ribozyme-mediated, gene “knockdown” strategy to effectively inhibit the expression of targeted gene in the developing zebrafish embryo: Proc. Natl. Acad. Sci. USA, 94:13777-13781 (1997). Returning to the subject invention, one could create a ribozyme delivery system with a strong promoter like cytomegalovirus (“CMV”) or an RNA pol III driven construct that could deliver ubiquitous high expression and a strong transcript size to inhibit gene function of hg in all organs. The ribozyme approach, to reduce the effect or “knockdown” hg, applied in mice or in a model organism like zebrafish (Xie et al., supra) can result in the rapid identification of the function of hg. Since our hg clone B308A-6-1 appears to be highly conserved across species, for the ribozyme strategy only a short sequence of target DNA is necessary; it would be feasible to use conserved regions of B308A-6-1 to predict targets for sequence determination in any other species.

It should be very feasible to use these regions of high homology to isolate the other species' hg genes by high or medium stringency hybridization, or by the polymerase chain reaction. One is able to isolate, by polymerase chain reaction, a fragment of DNA coding for hg or hg family members when using primers of degenerate sequence.

Thus, an aspect of this invention is to use oligonucleotide probes to detect DNA sequences complementary to the probes in a mixture of DNA sequences, or to select oligonucleotide primers preferably consisting of at least 20 contiguous nucleotides from SEQ ID NOS:1 or 3, or preferably at least 20 contiguous nucleotides complementary to these sequences. For example, among the PCR primers that are markers of the hg region and that have been used to amplify the STSs shown in the physical map of FIG. 1 are the following single stranded oligonucleotide sequences:

| | |
|---|---|
| TGGAAGCCAGAGACAAGCAG | SEQ ID NO:5 |
| AGAAATGGAAGCCAGAGACAA | SEQ ID NO:6 |
| CTTTTGACACCTTCCTCGATTC | SEQ ID NO:7 |
| CTCAAACCACAGGCCTCCGGA | SEQ ID NO:8 |

By “stringent conditions” we generally mean those which (1) employ low ionic strength and high temperature for washing, for example, 0.15M NaCl/0.015M sodium citrate/0.1% NaDodSO₄ at 50° C., or (2) use during hybridization a denaturing agent such as formamide, for example, 50% (vol/vol) formamide with 0.1% bovine serum albumin/0.1% Ficoll/0.1% polyvinylpyrrolidone/60 mM sodium phosphate buffer at pH 6.5 with 750 mM NaCl, 75 mm sodium citrate at 42° C. High-stringency conditions, for example, can be performed according to standard protocols (Ausubel et al., Current Protocols in Molecular Biology, Green Publishing Associates & Wiley Interscience, New York, 1987).

A number of applications for hg are suggested from its pharmacological (biological activity) properties.

The hg cDNA should be useful as a diagnostic tool, such as through use of antibodies in assays for proteins in cell lines or use of oligonucleotides as primers in a PCR test to amplify those with sequence similarities to the oligonucleotide primer, and to see how much hg is present.

By “substantial similarity” when referring to proteins, we mean that hg from different species, or hg family members within a species, have stretches of 10 consecutive amino acids or more having 80% identity in amino acid sequence.

Similarity at the protein level means an ability of a subject protein to compete with hg for binding to receptors or other interacting proteins and some (but not all) monoclonal antibodies raised against hg epitopes.

Hg, of course, might act upon its target cells via its own receptor. Hg, therefore, provides the key to isolate this receptor. Since many receptors mutate to cellular oncogenes, the hg receptor should prove useful as a diagnostic probe for certain tumor types. Thus, when one views hg as ligand in complexes, then complexes in accordance with the invention include antibody bound to hg, antibody bound to peptides derived from hg, hg bound to its receptor, or peptides derived from hg bound to its receptor or other factors. Mutant forms of hg, which are either more potent agonists or antagonists, are believed to be clinically useful. Such complexes of hg and its binding protein partners will find uses in a number of applications.

Practice of this invention includes use of an oligonucleotide construct comprising a sequence coding for hg and for a promoter sequence operatively linked to hg in a mammalian or a viral expression vector. Expression and cloning vectors contain a nucleotide sequence that enables the vector to replicate in one or more selected host cells. Generally, in cloning vectors this sequence is one that enables the vector to replicate independently of the host chromosomes, and includes origins of replication or autonomously replicating sequences.

Practice of this invention includes preparation and uses of diagnostic or therapeutic agents. That is, hg preparations could be useful as standards in assays for hg and in competitive-type receptor binding assays when labelled with radioiodine, enzymes, fluorophores, spin labels, and the like. Therapeutic formulations of hg could be prepared for storage by mixing hg having the desired degree of purity with optional physiologically acceptable carriers, excipients or stabilizers, in the form of lyophilized cake or aqueous solutions.

Polyclonal antibodies to hg could be raised in animals by multiple subcutaneous (sc) or intraperitoneal (ip) injections of hg and an adjuvant. It may be useful to conjugate hg or a fragment containing the target amino acid sequence to a protein which is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR.

Monoclonal antibodies are prepared by recovering spleen cells from immunized animals and immortalizing the cells in conventional fashion, e.g. by fusion with myeloma cells or by EB virus transformation and screening for clones expressing the desired antibody.

Hg antibodies could be useful in diagnostic assays for hg or its antibodies and to identify family members. In one embodiment of a receptor binding assay, an antibody composition which binds to all or to a selected plurality of members of the hg family could be immobilized on an insoluble matrix, the test sample contacted with the immobilized antibody composition in order to adsorb all hg family members, and then the immobilized family members contacted with a plurality of antibodies specific for each member, each of the antibodies individually identifiable as specific for a predetermined family member, as by unique labels such as discrete fluorophores or the like. By determining the presence and/or amount of each unique label, the relative proportion and amount of each family member can be determined.

Hg antibodies also could be useful for the affinity purification of hg from recombinant cell culture or natural sources. Hg antibodies that do not detectably cross-react with other growth factors could be used to purify hg free from these other family members.

It is to be understood that while the invention has been described above in conjunction with preferred specific embodiments, the description and examples are intended to illustrate and not limit the scope of the invention, which is defined by the scope of the appended claims.

We claim:

1. An isolated nucleic acid molecule encoding a gene product which, when knocked out, results in a high growth phenotype.

2. An isolated cDNA having the nucleotide sequence of SEQ ID NO:1.

3. A construct comprising the nucleotide sequence of SEQ ID NO:1 operatively linked with an expression vector.

4. A transformant obtained by introducing the construct of claim 3 into a host.

5. A probe or primer, comprising:
at least 20 contiguous nucleotides selected from SEQ ID NO:1 or at least 20 contiguous nucleotides complementary to SEQ ID NO:1, SEQ ID NO:2, or SEQ ID NO:3.

6. Isolated monoclonal or polyclonal antibodies to SEQ ID NO:4.
